(19) United States
(12) Patent Application Publication
(10) Pub. No.: US 2020/0005933 A1
(43) Pub. Date: Jan. 2, 2020

(54) PERSONAL INFORMATION OBJECT

(71) Applicant: MEIDC LLC, Huntington Beach, CA (US)

(72) Inventor: Michele Castellano, Huntington Beach, CA (US)

(21) Appl. No.: 16/269,135

(22) Filed: Feb. 6, 2019

Related U.S. Application Data
(62) Division of application No. 14/991,967, filed on Jan. 10, 2016, now abandoned.
(60) Provisional application No. 62/101,616, filed on Jan. 9, 2015.

(51) Int. Cl. G16H 40/20; G16H 10/60

(52) U.S. Cl. CPC G16H 40/20; G16H 10/60

(57) ABSTRACT

A personal information object comprises at least one barcode. The at least one barcode is encoded to contain at least a portion of personal data associated with an owner of the personal information object. The portion of personal data comprises at least one of the following declarations: a waiver of privacy rights, an advanced directive declaration, a Do Not Resuscitate (DNR) declaration, an Allow a Natural Death (AND) declaration; and a Do Not Incubate (DNI) declaration. The personal information object further comprises a signature for authorization of at least one of the declarations.

### CROSS-REFERENCE TO RELATED APPLICATIONS

This application claims the benefit of U.S. Provisional Application No. 62/101,616, filed Jan. 9, 2015, which is hereby incorporated by reference in its entirety.

### BACKGROUND

Personal identification cards may not provide a sufficient amount of personal information in an emergency situation. Medical information cards may not provide first responders and/or medical staff with all of the personal information needed to provide the best care for patients. Some medical information cards may provide a link or a barcode comprising a link to additional personal information. However, the additional personal information is often presented and/or transmitted in a format that may be incompatible with the format used by first responders and/or medical staff. Furthermore, accessing the additional personal information requires network connectivity which may not be available when needed. Although some personal information may be provided through a mobile device, first responders may not be able to locate the mobile device in an emergency or access all of the personal information required if the mobile device is locked. What is needed is an improved personal information object.

### BRIEF DESCRIPTION OF THE SEVERAL VIEWS OF THE DRAWINGS

FIG. 1 is an example block diagram showing a personal information card as per an aspect of an embodiment.

FIG. 2 is an example block diagram showing a personal information card as per an aspect of some of the various embodiments.

FIG. 3 illustrates one side of an example personal information card as per an aspect of some of the various embodiments.

FIG. 4 illustrates one side of an example personal information card as per an aspect of some of the various embodiments.

FIG. 5 is an example flow diagram of sending instructions to print a personal information card as per an aspect of an embodiment.

FIG. 6 is an example block diagram showing various components of a personal information card processing system as per an aspect of some of the various embodiments.

FIG. 7 is an example flow diagram of sending instructions to modify a personal information article as per an aspect of an embodiment.

FIG. 8 is an example block diagram showing various components of a personal information article processing system as per an aspect of some of the various embodiments.

FIG. 9 is an example flow diagram of producing a personal information object as per an aspect of some of the various embodiments.

FIG. 10 is an example flow diagram of producing a personal information object as per an aspect of some of the various embodiments.

FIG. 11A, FIG. 11B, FIG. 11C, and FIG. 11D illustrate examples of personal information articles as per aspects of some of the various embodiments.

FIG. 12 is a block diagram of a computing environment in which aspects of embodiments of the present invention may be practiced.

### DETAILED DESCRIPTION OF EMBODIMENTS

Embodiments of the present invention relate to personal information objects.

Some of the various embodiments may include a personal information card. The personal information card may comprise personal data associated with a card holder. The personal information card may further comprise at least one barcode. At least one of the at least one barcode may comprise a two-dimensional barcode. At least one of the at least one barcode may comprise a three-dimensional barcode. The at least one barcode may be encoded to contain at least a portion of the personal data associated with the card holder. The portion of personal data may comprise declaration(s). The declaration(s) may comprise at least one of a waiver of privacy rights, an advanced directive declaration, a Do Not Resuscitate (DNR) declaration, an Allow a Natural Death (AND) declaration, a Do Not Incubate (DNI) declaration, combinations thereof, and/or the like. The privacy rights may be associated with the Health Insurance Portability and Accountability Act (HIPAA) Privacy Rule. Persons skilled in the art will recognize that the privacy rights may be associated with other rights, rules, acts, and/or laws associated with personal and/or medical information. The declaration(s) may also comprise a refusal to declare at least one waiver, directive, order, combinations thereof, and/or the like. The declaration(s) may comprise information representing the decision by the user to declare and/or refuse to declare at least one waiver, directive, order, combinations thereof, and/or the like. For example, the declaration(s) may comprise a bit or flag indicating a choice made by the card holder for at least one of a waiver of privacy rights, an advanced directive declaration, a Do Not Resuscitate (DNR) declaration, an Allow a Natural Death (AND) declaration, a Do Not Incubate (DNI) declaration, combinations thereof, and/or the like. According to some of the various embodiments, at least one of the declaration(s) may comprise a contact name and contact information of a person or representative designated by the card holder. At least one of the declaration(s) may be distinct for the state of residence of the card holder. The personal information card may further comprise a signature from the card holder or a legal guardian, medical durable power of attorney, relative, doctor, attorney, friend, combinations thereof, and/or the like of the card holder for authorization of at least one of the declaration(s). The signature may comprise a bit or flag indicating an acceptance of a waiver of privacy rights made by the card holder, and/or confirmation of an advanced directive declaration, a Do Not Resuscitate (DNR) declaration, an Allow a Natural Death (AND) declaration, a Do Not Incubate (DNI) declaration, combinations thereof, and/or the like. The personal information card may further comprise a signature relationship to the card holder. For example, the signature relationship may comprise self, legal guardian, spouse, parent, child, doctor, attorney, friend, sibling, combinations thereof, and/or the like.

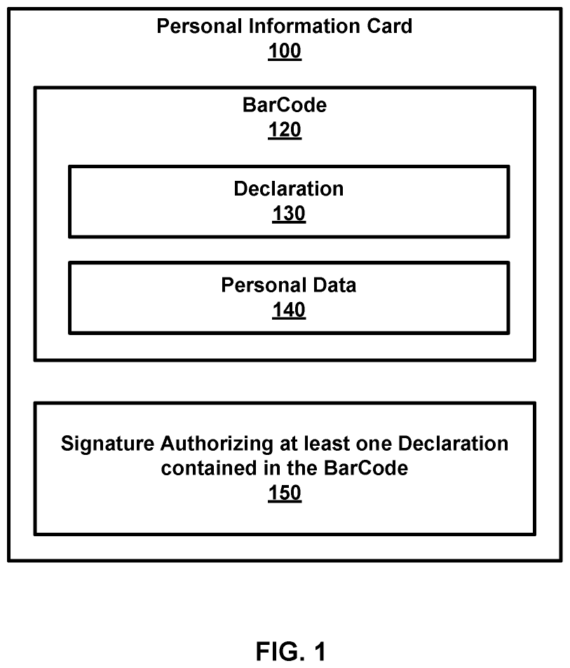

FIG. 1 is an example block diagram showing a personal information card 100 as per an aspect of an embodiment. Personal Information Card 100 may comprise BarCode 120 and Signature 150. BarCode 120 may be encoded to contain Declaration 130 and Personal data 140.

According to some of the various embodiments, the personal information card may be temporary. Alternatively, the personal information card may be permanent. The personal information card may be printed by the user. By way of example and not limitation, the permanent personal information card may be delivered via mail or parcel service. The permanent personal information card may be a replacement for a temporary personal information card.

According to some of the various embodiments, a personal information card may comprise a watermark. The watermark may be configured to designate that the personal information card is temporary. The watermark may be configured to cover at least a portion of at least one barcode. The at least one barcode may be presented on a display or printed on a personal information card. The watermark may be configured to prevent successful scanning of at least one barcode. By way of example and not limitation, successful scanning may be prevented by covering at least a portion of the at least one barcode with at least a portion of the watermark, and/or altering at least a portion of the at least one barcode with at least a portion of the watermark. According to some of the various embodiments, the watermark may be superimposed over a barcode on a display or on a temporary card until a purchase is made. The watermark may be superimposed over a barcode on a display or a temporary card if personal data is changed. The watermark may designate that at least a portion of the personal data is outdated.

According to some of the various embodiments, the personal information card may be flexible. The personal information card may comprise at least one adhesive on one side of the card. An adhesive patch, decal, sticker, material, combinations thereof, and/or the like may comprise the personal information card.

According to some of the various embodiments, the personal information card 100 may be associated with a card template. The card template may comprise a sponsor designator, an advertiser designator, a non-profit designator, a confirmation of a donation, a promotional offer, a URL, an advertisement, combinations thereof, and/or the like. By way of example and not limitation, a designator may be a name, a logo, a slogan, a picture of a product, combinations thereof, and/or the like. The promotional offer may comprise a coupon, a coupon code, reorder instructions for one or more additional cards, combinations thereof, and/or the like.

According to some of the various embodiments, the personal information card 100 may comprise a biometric identifier of the card holder. By way of example and not limitation, the biometric identifier may be associated with a fingerprint, a thumbprint, palm veins, palm print, hand geometry, iris recognition, a retina scan, a DNA sequence or subsequence, face recognition, combinations thereof, and/or the like. The personal information card 100 may comprise a photo of the card holder. The photo may be associated with a creation date this is encoded and/or displayed with the photo. Personal data included on the personal information card, and/or the format of at least a portion of the personal data, may be specific to a state or local jurisdiction. The personal information card 100 may also comprise card information. The card information may comprise a card creation data, a card print date, a symbol representing a portion of the personal data, an acronym representing a portion of the personal data, combinations thereof, and/or the like.

According to some of the various embodiments, at least one barcode may be formatted as a Quick Response Code. At least one barcode may be formatted as a Portable Data File. The at least one barcode may comprise an encoded signature.

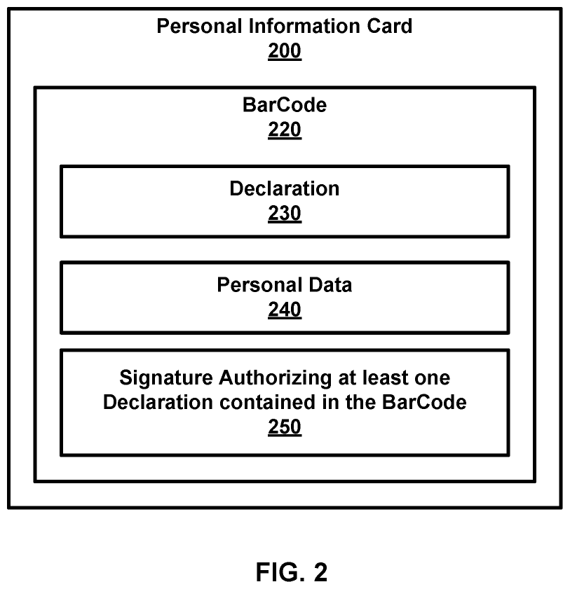

FIG. 2 is an example block diagram showing a personal information card 200 as per an aspect of some of the various embodiments. Personal Information Card 200 may comprise BarCode 220. BarCode 220 may be encoded to contain at least one Declaration 230, Personal data 240, and Signature 250.

According to some of the various embodiments, personal data 240 may comprise personal attribute information. Personal attribute information may comprise an address, a blood type, a body weight, a date of birth, an eye color, a gender, a hair color, a height, a name, a religious preference, a thumb or finger print identifier, a military status, a veteran status, a description of one or more pets, a registration number associated with one or more pets, at least one note, combinations thereof, and/or the like. By way of example and not limitation, a thumb or finger print identifier may comprise a print, an image, a hyperlink, an encoding, a digital ID, combinations thereof, and/or the like.

According to some of the various embodiments, personal data 240 may comprise contact information. Contact information may comprise emergency contact information, a health care provider contact, a primary care physician contact, contact information for a relative, combinations thereof, and/or the like. Emergency contact information may comprise a name, a phone number, an email address, a relational status, combinations thereof, and/or the like. The health care provider contact may comprise a name, a phone number, an email address, combinations thereof, and/or the like. Contact information for a relative may comprise a name, a phone number, an email address, combinations thereof, and/or the like. By way of example and not limitation, a relative may comprise a father, a mother, a guardian, a spouse, a sibling, next of kin, a child, combinations thereof, and/or the like.

According to some of the various embodiments, personal data 240 may comprise association information. Association information may comprise an American Red Cross donor identification number. Donate Life America registration, a Healthbook™ ID, International SOS membership information, Smart Traveler Enrollment Program account information, a hospital preference, at least a portion of a social security number, a state issued identification number, combinations thereof, and/or the like. The Smart Traveler Enrollment Program is an example of a traveler program and is a service of the Bureau of Consular Affairs, a bureau of the United States Department of State.

According to some of the various embodiments, personal data 240 may comprise legal status information. Legal status information may comprise a citizenship, a citizenship status, a marital status, a parent relationship status, a registered arson offender status, a registered narcotics offender status, a registered sex offender status, combinations thereof, and/or the like. By way of example and not limitation, a citizenship status may comprise a resident, an alien, a visa, a visa type, an employment authorization document, combinations thereof, and/or the like. The marital status may comprise single, married, divorced, widowed, combinations thereof, and/or the like. The parent relationship status may comprise foster, adopted, and/or the like. An example of a registered arson offender status may be found in California Penal Code Sections 451 and 452. An example of a registered narcotics offender status may be found in California Health and Safety Code Section 11590. An example of a registered sex offender status may be found in California Penal Code Section 290.

According to some of the various embodiments, personal data 240 may comprise medical association information. Medical association information may comprise an implant registration number, a health care policy number, a Medicaid number, a Medicare number, a state sponsored medical program number, combinations thereof, and/or the like. An example of a state sponsored medical program may be Medi-Cal. Examples of implants include breast implants, stints, joint replacements, dental implants, combinations thereof, and/or the like.

According to some of the various embodiments, personal data 240 may comprise medical condition information. Medical condition information may comprise allergy information, an eye glass need statement, a hard of hearing statement, hearing impairment, information relating to metal present in a body, a major health concern, a recreational drug user statement, a special need, instructions for treatment of any of the aforementioned medical conditions, combinations thereof, and/or the like. Allergy information may, for example, comprise at least one allergy, at least one allergic reaction type, at least one allergy severity, combinations thereof, and/or the like. Information relating to metal present in a body may, for example, comprise location, type of metal, shape, combinations thereof, and/or the like. A major health concern may, for example, comprise angina pectoris, arrhythmia, artificial heart information, asthma, atrial fibrillation, a history of cancer, cardiovascular disease, at least one cerebrovascular accident, Chronic Obstructive Pulmonary Disease (COPD), Congestive Heart Failure (CHF), diabetes, dyslipidemia, emphysema, a history of epilepsy, gestational diabetes, at least one prior heart attack, hypoglycemia, hypertension, kidney disease. Organic Brain Syndrome (OBS), a Prostate-specific Antigen PSA level, pulmonary fibrosis, sarcoidosis, a history of seizures, at least one prior stroke, at least one prior tracheotomy, Transient Ischemic Attack (TIA), combinations thereof, and/or the like. A special need may, for example, comprise Alzheimer's, Autism, Dementia, Memory loss. Multiple Sclerosis, combinations thereof, and/or the like.

According to some of the various embodiments, personal data 240 may comprise medical history information. Medical history information may comprise a medical event history, a psychological history, a family medical history, a genetic disorder, combinations thereof, and/or the like. The medical event history may comprise surgery information, at least one date, combinations thereof, and/or the like.

According to some of the various embodiments, personal data 240 may comprise medical status information. Medical status information may comprise an artificial heart status, an autistic status, a cancer survivorship status, a contact lens designation, a diabetic status, a Down syndrome status, a heart transplant status, medication information, a pregnancy status, a smoker status, a tetanus shot date, vaccine information, a vaccine date, at least one diagnostic code, at least one classification, combinations thereof, and/or the like. The diabetic status may comprise a type of diabetes. Medication information may comprise current medications (e.g. over the counter and/or prescription), medication duration, at least one medication code, at least one classification, medication allergies, allergic reactions, combinations thereof, and/or the like. The pregnancy status may comprise a month, a trimester, a due date, combinations thereof, and/or the like. Vaccine information may comprise at least one received date, at least one vaccination type, combinations thereof, and/or the like. An example of the at least one diagnostic code is the Healthcare Common Procedure Coding System (HCPCS). An example of the at least one classification may comprise a revision of the International Statistical Classification of Diseases and Related Health Problems (ICD).

According to some of the various embodiments, personal data 240 may comprise financial account information. Financial account information may comprise mortgage account information, banking account information, credit account information, at least one reference to a financial account, combinations thereof, and/or the like. By way of example and not limitation, a reference may comprise at least one hyperlink, at least one contact name, at least one contact phone number, at least one contact address, at least one contact e-mail address, combinations thereof, and/or the like.

According to some of the various embodiments, personal data 240 may comprise declaration 230 information. Declaration 230 information may comprise an anatomical gift declaration, a declaration to waive at least one privacy right, a directive, a donor wish, a do not incubate declaration, a do not resuscitate declaration, an Allow a Natural Death declaration, a signature referring to at least one of the aforementioned declaration information, combinations thereof, and/or the like. An anatomical gift declaration may comprise registration information applicable to the card holder. The registration may refer to at least one organ, at least one cornea, skin, blood, at least one tissue, combinations thereof, and/or the like. At least one registration may be with a state of residence of the card holder. Alternatively, the registration may be associated with the Uniform Anatomical Gift Act (UAGA). At least one of the declaration(s) may comprise a reference. By way of example and not limitation, at least one of the declaration(s) may be configured to give consent and/or remove consent to a future action taken by a bystander, a first responder, a medical professional, a doctor, a legal professional, a lawyer, a relative, combinations thereof, and/or the like.

According to some of the various embodiments, a signature 250 may comprise a written signature, an encoded signature, a reference to a signature, combinations thereof, and/or the like. By way of example and not limitation, an encoded signature may comprise a digital signature, an e-signature, touch point data, a digital document, a digital file, a hyperlink, a virtual sampler bank (vsb) file, a bitmap (bmp) image file, joint photographic experts group (jpeg) file, a portable network graphics file (png), combinations thereof, and/or the like. Persons skilled in the art(s) will recognize many more examples of image files which may comprise an encoded image. The reference to a signature may comprise a digital document, a third party, a physical location, a third party database, combinations thereof, and/or the like.

According to some of the various embodiments, a method of printing a personal information card may comprise receiving personal data. The personal data may be received via at least one processor. Personal data may be associated with a card holder. The method of printing a personal information card may further comprise obtaining a signature from a user for authorization of at least one of the declaration(s). At least one of the declaration(s) may, for example, be presented to a user along with identification verification. In this example, the signature may be obtained to verify identity and authorize at least one of the declaration(s). Obtaining a signature may comprise requesting a signature and receiving a signature. At least one of the declaration(s) may comprise a waiver of privacy rights, an advanced directive declaration, a Do Not Resuscitate (DNR) declaration, an Allow a Natural Death (AND) declaration, a Do Not Incubate (DNI) declaration, combinations thereof, and/or the like. At least one of the declaration(s) may be distinct for the state of residence of the anticipated card holder. At least one of the declaration(s) may comprise a reference. The reference may be designated by the user or the anticipated card holder. The method of printing a personal information card may further comprise presenting at least a portion of the personal data on a display. The method of printing a personal information card may further comprise sending at least a portion of personal data to a printer with instructions to print a personal information card. Alternatively, the instructions to print a personal information card may be stored in an electronic file. The file may be employed to transmit the instructions to print to a printing device. The printer may comprise a virtual printer. Some examples of a virtual printer comprise CutePDF Writer, RedMon, Microsoft XPS Document Writer. Adobe Distiller, combinations thereof, and/or the like. The printer may be configured to generate an electronic file. The electronic file may, for example, comprise printing instructions, barcode generation instructions, at least one image, an image file, combinations thereof, and/or the like. According to some of the various embodiments, the method of printing a personal information card may further comprise sending instructions to print at least one additional personal information card. The method of printing a personal information card may further comprise sending instructions to print a card template. The personal information card may comprise at least one barcode. The at least one barcode may be encoded to contain at least a portion of the personal data. The portion of the personal data may comprise at least one of the declaration(s).

Some of the various embodiments may include a non-transitory computer readable medium comprising computer readable instructions. The computer readable instructions may be configured to cause at least one processor to receive personal data associated with an anticipated card holder. By way of example and not limitation, the anticipated card holder may be a user of the computer readable instructions attempting to order and/or print a personal information card. Alternatively, the user may be attempting to order and/or print a personal information card for at least one other person. The user may be a relative, legal guardian, medical durable power of attorney, doctor, attorney, friend, combinations thereof, and/or the like of the anticipated card holder. The user may be a business administrator, an organization administrator, a head of household, combinations thereof, and/or the like associated with the anticipated card holder. An example of a business administrator is a person who works for a business. An example of an organization administrator is a person who works for and/or volunteers for an organization. According to some of the various embodiments, validation of an identity of the anticipated card holder may be required. By way of example and not limitation, validation of identity may employ a biometric scan, a barcode scan, an identification number, a social security number, a financial account number, combinations thereof, and/or the like.

According to some of the various embodiments, the computer readable instructions may be configured to cause at least one processor to present at least a portion of the personal data on a display. The computer readable instructions may be configured to cause at least one processor to present at least one of the declaration(s) on a display. At least one of the declaration(s) may be distinct for the state of residence of the anticipated card holder. The computer readable instructions may also be configured to cause at least one processor to obtain a signature from the user for authorization of at least one of the declaration(s). At least one of the declaration(s) may be associated with the anticipated card holder. The computer readable instructions may be further configured to cause at least one processor to send at least a portion of the personal data to a printer with instructions to print a personal information card.

According to some of the various embodiments, personal data may be received from a data file, a database, a web based form accessible over a computer network, a data entry form configured to accept data directly from a user, combinations thereof, and/or the like. Personal data may be stored in at least one data file. By way of example and not limitation, the at least one data file may be stored on a computing device, a mobile device, at least one server accessed over the Internet, combinations thereof, and/or the like. The personal data may be stored in at least one data file associated with a unique user. The computer readable instructions may be further configured to require user authentication to access the data file. User authentication may be restricted to the user associated with the personal data. The computer readable instructions may be further configured to enable a user to edit the personal data. The computer readable instructions may be further configured to encrypt the personal data.

According to some of the various embodiments, computer readable instructions may be configured to cause at least one processor to present a user interaction item on a display. The user interaction item may comprise at least one clickable button, at least one swipeable item, at least one speech recognition icon, combinations thereof, and/or the like. Clickable buttons and swipeable items comprise User Interface (UI) items presented on, for example, touch screen displays. According to some of the various embodiments, the user interaction item may be configured to present emergency contact information, health care provider information, insurance provider information, relative contact information, financial donation information, donor information, order information for at least one personal information card, combinations thereof, and/or the like. The user interaction item may be presented as a button, an icon, a symbol, and/or the like. The user interaction item may comprise a link to a webpage or instructions further configured to place a phone call to a number included in any of the aforementioned information. The webpage may be configured to present information on determining blood type, donor wishes, capturing a thumb or finger print, producing a barcode, combinations thereof, and/or the like. By way of example and not limitation, the barcode may be formatted as a Quick Response (QR) code, a Portable Data File (PDF) 417 barcode, a matrix barcode, and/or the like. The user interaction item may be configured to import at least a portion of the personal data. By way of example and not limitation, at least a portion of the personal data may be imported from a memory location, a file, a database, a website, a web based form, a data entry form, combinations thereof, and/or the like. At least a portion of the personal data may be scanned from a barcode residing on a driver's license, an identification card, an identification item, a display, combinations thereof, and/or the like.

According to some of the various embodiments, the signature may comprise a digital signature, an imported image, a captured image, an encoded signature, an imported file, a video testimony, combinations thereof, and/or the like. By way of example and not limitation, the video testimony may be embedded in a barcode. Alternatively, the video testimony may be accessed through a reference embedded in a barcode. According to some of the various embodiments, the computer readable instructions may be further configured to enable the user to opt out of the signature.

According to some of the various embodiments, the printer may be accessed via a local connection, a wireless connection, a cable, the Internet, an intranet, a network, a wireless network, a wired network connection, combinations thereof, and/or the like. By way of example and not limitation, a wireless connection may comprise Near-Field Communications (NFC), Bluetooth, Infrared (IR), Radio Frequencies (RF), combinations thereof, and/or the like. By way of example and not limitation, a cable may be configured as a Universal Serial Bus (USB) cable, a printer cable, a twisted pair of cables, a Digital Subscriber Line (DSL), a coax cable, a fiber optic cable, combinations thereof, and/or the like. The instructions to print may further comprise instructions to print at least one additional personal information card. The instructions to print may further comprise instructions to print a card template associated with the personal information card. The instructions to print may relate to a temporary personal information card and/or a permanent personal information card. The instructions to print may further comprise instructions to print a watermark on the personal information card.

According to some of the various embodiments, the instructions to print the personal information card may comprise instructions to cause a printing device to print declaration(s). At least one of the declaration(s) may comprise a waiver of privacy rights, an advanced directive declaration, a do not resuscitate declaration, an Allow a Natural Death declaration, a do not incubate declaration, combinations thereof, and/or the like. The instructions to print the personal information card may comprise instructions to cause a printing device to print a signature of the anticipated card holder. The instructions to print the personal information card may also comprise instructions to cause a printing device to print a biometric identifier of the anticipated card holder. The instructions to print the personal information card may comprise instructions to cause a printing device to print a photo of the anticipated card holder. The instructions to print the personal information card may be specific to a state or local jurisdiction. The instructions to print the personal information card may further comprise card information.

According to some of the various embodiments, the instructions to print the personal information card may comprise instructions to cause a printing device to print the at least one barcode. The at least one barcode may be encoded to contain at least a portion of the personal data associated with the anticipated card holder. The portion of the personal data may comprise at least one of the declaration(s). The at least one barcode may comprise an encoded signature of the user and/or the anticipated card holder.

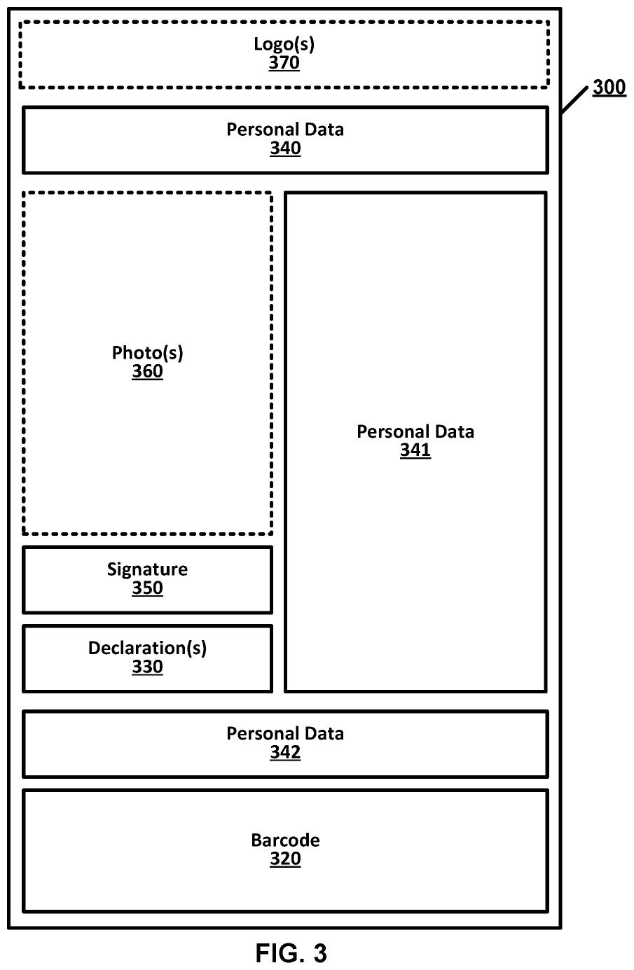

FIG. 3 illustrates one side of an example personal information card as per an aspect of some of the various embodiments. Personal Information Card 300 may comprise at least one Declaration 330 and Signature 350. Personal Information Card 300 may comprise Personal Data 340, Personal Data 341, and Personal Data 342. Personal Information Card 300 may comprise BarCode 320. BarCode 320 may be encoded to contain Declaration 330. Signature 350, and/or at least a portion of the personal data (e.g. 340, 341, and 342). Furthermore, Barcode 320 may be encoded to contain personal data that is not presented in Personal Data 340, Personal Data 341, and/or Personal Data 342. Personal Information Card 300 may comprise at least one Logo 370. Personal Information Card 300 may comprise at least one Photo 360.

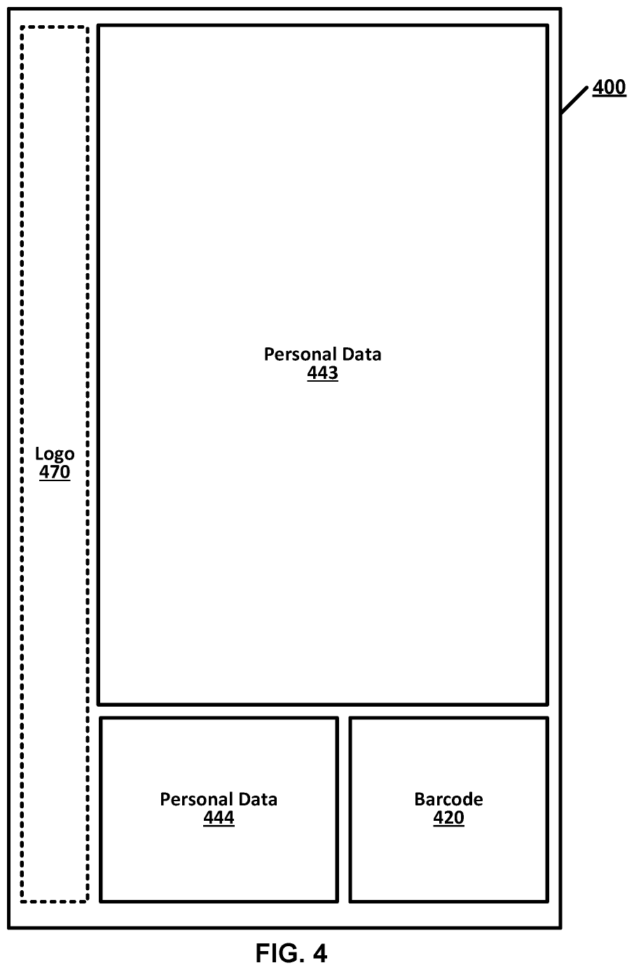

FIG. 4 illustrates another side of an example personal information card as per an aspect of some of the various embodiments. Personal Information Card 400 may comprise BarCode 420, Personal Data 443, and Personal Data 444. BarCode 420 may be encoded to contain at least a portion of the personal data (e.g. 443, and 444). Furthermore. Barcode 420 may be encoded to contain personal data that is not presented in Personal Data 443, and/or Personal Data 444. Personal Information Card 400 may comprise at least one Logo 470.

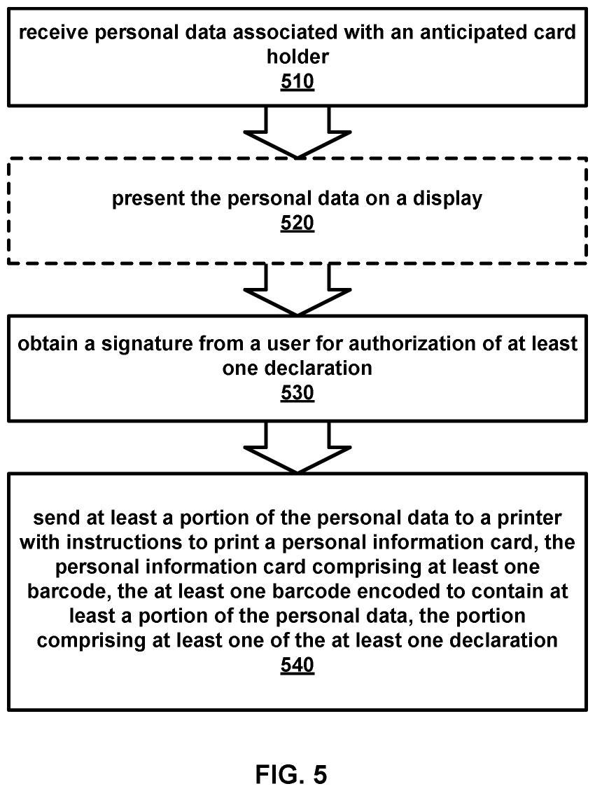

FIG. 5 is an example flow diagram of sending instructions to cause a printing device to print a personal information card as per an aspect of an embodiment. Personal data associated with an anticipated card holder may be received at block 510. Personal data may be presented on a display at block 520. At block 530, a signature from a user and/or anticipated card holder may be obtained for authorization of at least one of the declaration(s). At least a portion of the personal data may be sent to a printer with instructions to print a personal information card at block 540. The personal information card may, for example, comprise at least one barcode. The at least one barcode may be encoded to contain at least a portion of the personal data. The portion of personal data may comprise at least one of the declaration(s).

According to some of the various embodiments, the computer readable instructions may be further configured to cause at least one processor to present the at least one barcode on a display. The computer readable instructions may reside on a mobile device and/or a wearable device. The computer readable instructions may be further configured to cause at least one processor to present the at least one barcode on the display based at least in part on a signal from at least one motion sensor, gravity sensor, impact sensor, accelerometer, combinations thereof, and/or the like. For example, a signal from an accelerometer may indicate a mobile device suffered an impact acute enough to likely cause injury to the user of the mobile device. Upon receiving this signal, the at least one processor may be instructed to present the at least one barcode on the display. According to some of the various embodiments, the computer readable instructions may be further configured to cause at least one processor to present an option to the user to alert an emergency contact, alert at least one emergency service, contact an emergency contact, contact at least one emergency service, combinations thereof, and/or the like. According to some of the various embodiments, the computer readable instructions may be further configured to cause at least one processor to present an option to the user to cancel presentation of the at least one barcode. For example, if the mobile device is dropped inadvertently, an accelerometer may indicate a mobile device suffered an impact acute enough to likely cause injury to the user of the mobile device. Upon receiving this signal, the at least one processor may be instructed to present the at least one barcode on the display. However, in this example, the user may be presented with an option to cancel presentation of the at least one barcode. The option may, for example, comprise at least one user interaction item, a plurality of user interaction items, a request for a password, a request for a code, a biometric scan, combinations thereof, and/or the like. By way of example and not limitation, the computer readable instructions may be configured to cause at least one processor to require interaction with the plurality of user interaction items in a predetermined sequence. According to some of the various embodiments, personal information, at least one of the declaration(s), a signature, combinations thereof, and/or the like may be presented on a display with the at least one barcode.

According to some of the various embodiments, the computer readable instructions may integrate with emergency call screen instructions configured to cause at least one processor to present an emergency call screen. Integration with emergency call screen instructions may, for example, comprise presenting an icon when the mobile device screen is in a locked state. By way of example and not limitation, the icon may link to a wallpaper, application screen, backdrop, and/or the like comprising at least a portion of the personal data and/or at least one barcode. According to some of the various embodiments, the computer readable instructions may be configured to cause at least one processor to enable a first responder to bypass password protection of the mobile device to view at least a portion of the personal data and/or at least one barcode.

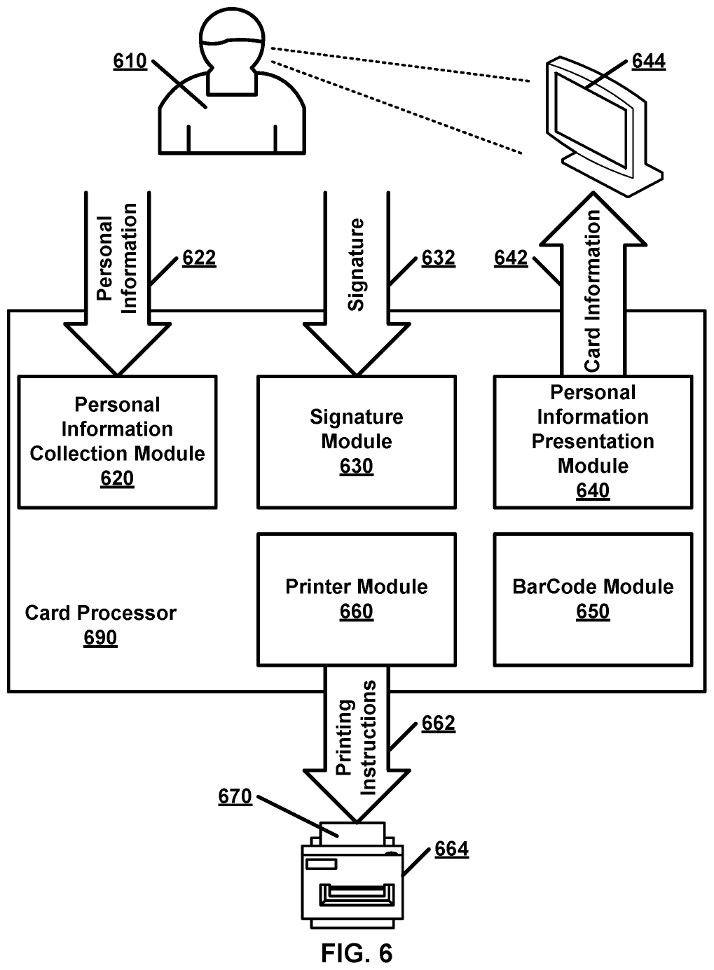

FIG. 6 is an example block diagram showing various components of a personal information card processing system as per an aspect of some of the various embodiments. Card Processor 690 may comprise Personal Information Collection Module 620, Signature Module 630, Card Information Presentation Module 640. BarCode Module 650, and Printer Module 660. Personal Information Collection Module 620 may receive Personal Information 622 from User 610. Signature Module 630 may receive Signature 632 from User 610. Card Information Presentation Module 640 may present Card Information 642 on Display 644. The Display 644 may be visible to User 610. BarCode Module 650 may create at least one barcode for at least one of the Card Information Presentation Module 640 and the Printer Module 660. Printer Module 660 may send Printing Instructions 662 to Printer 664. Printer 664 may be configured to print a Personal Information Card 670.

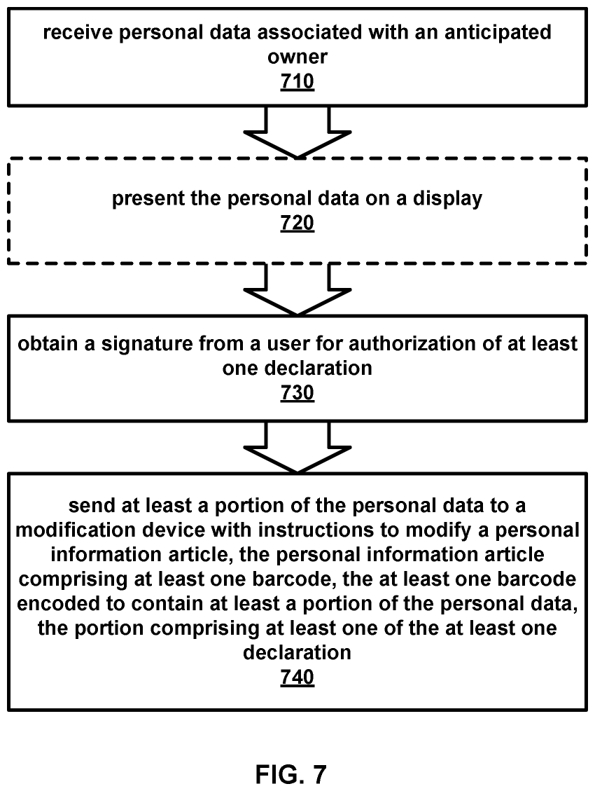

FIG. 7 is an example flow diagram of sending instructions to modify a personal information article as per an aspect of an embodiment. Personal data associated with an anticipated owner may be received at block 710. Personal data may be presented on a display at block 720. At block 730, a signature from a user and/or anticipated owner may be obtained for authorization of at least one of the declaration(s). At least a portion of the personal data may be sent to a modification device with instructions to modify a personal information article at block 740. The personal information article may, for example, comprise at least one barcode. The at least one barcode may be encoded to contain at least a portion of the personal data. The portion of personal data may comprise at least one of the declaration(s).

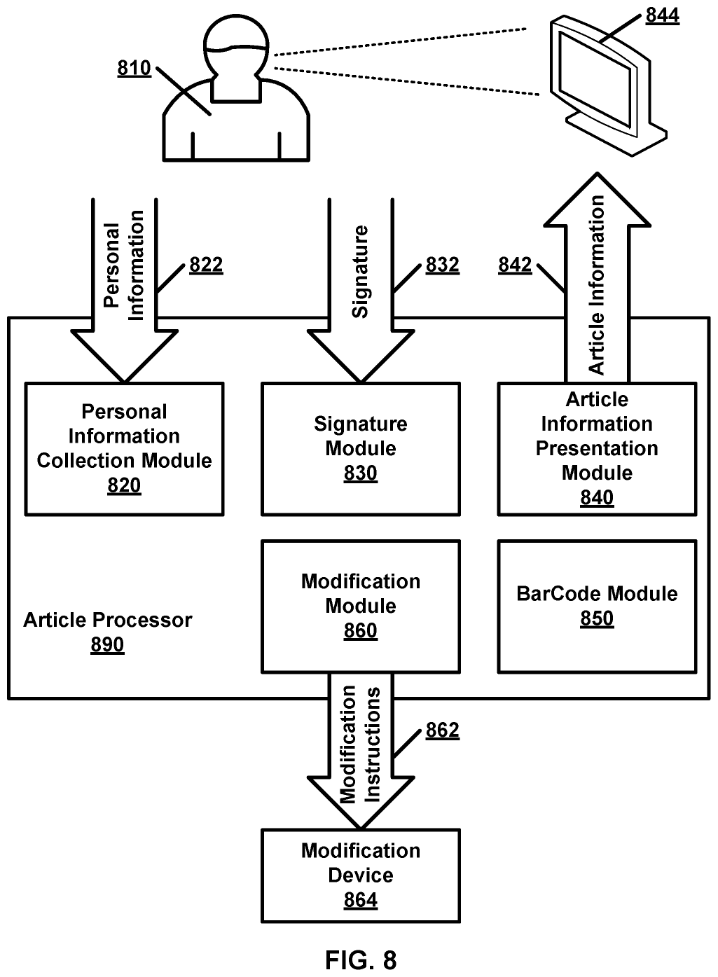

FIG. 8 is an example block diagram showing various components of a personal information article processing system as per an aspect of some of the various embodiments. Article Processor 890 may comprise Personal Information Collection Module 820, Signature Module 830, Article Information Presentation Module 840, BarCode Module 850, and Modification Module 860. Personal Information Collection Module 820 may receive Personal Information 822 from User 810. Signature Module 830 may receive Signature 832 from User 810. Article Information Presentation Module 840 may present Article Information 842 on Display 844. The Display 844 may be visible to User 810. BarCode Module 850 may create at least one barcode for at least one of the Article Information Presentation Module 840 and the Modification Module 860. Modification Module 860 may send Modification Instructions 862 to Modification Device 864. Modification Device 864 may be configured to modify a personal information article.

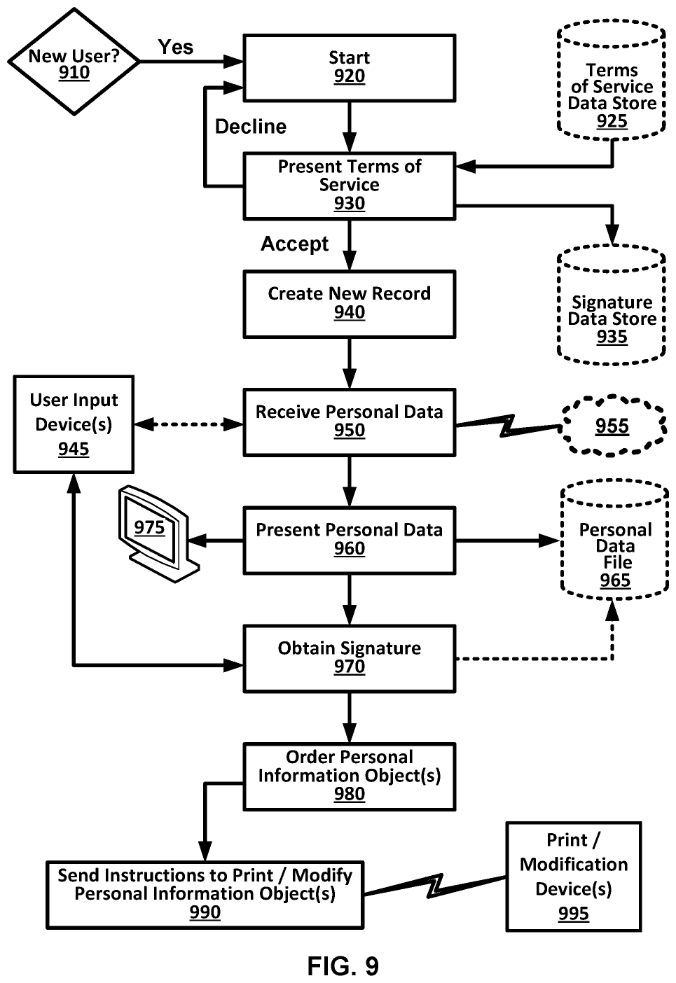

FIG. 9 is an example flow diagram of producing a personal information object as per an aspect of some of the various embodiments. Producing a personal information object may comprise determining if a user is a new user or an existing user at block 910. A new user process may be started at block 920. Terms of Service may be presented at block 930. The Terms of Service may, for example, comprise an End User License Agreement (EULA) and/or a Privacy Policy. The Terms of Service may be stored in a Terms of Service Data Store 925. Producing a personal information object may comprise returning new users who decline the Terms of Service back to start at block 920. Producing a personal information object may comprise creating a new record at block 940 for users who accept the Terms of Service. Acceptance of Terms of Service may comprise a user signature. The user signature may comprise an encoded signature. The signature may be recorded along with information that may be used to identify the user. The signature may be stored in a Signature Data Store 935. Producing a personal information object may comprise receiving personal data at block 950. At least a portion of the personal data may be received through at least one User Input Device 945. At least a portion of the personal data may be received from a remote device. The remote device may be associated with Cloud 955. Producing a personal information object may comprise presenting personal data at block 960. At least a portion of the personal data may be presented on Display 975. At least a portion of the personal data may be stored in a Personal Data File 965. The personal data file may, for example, be stored on a user device, on a detachable storage drive, in a personal data store located on at least one remote device, combinations thereof, and/or the like. Producing a personal information object may comprise obtaining a signature at block 970. Producing a personal information object may comprise presenting a privacy policy and/or at least a portion of the terms of service prior to obtaining a signature at block 970. The signature obtained at block 970 may, for example, authorize at least one declaration. User Input Device 945 may be employed to obtain a signature at block 970. Producing a personal information object may comprise accepting at least one order for at least one personal information object at block 980. Producing a personal information object may comprise sending instructions to print a personal information card and/or modify a personal information article at block 990. The instructions may be sent to at least one Print/Modification Device 995.

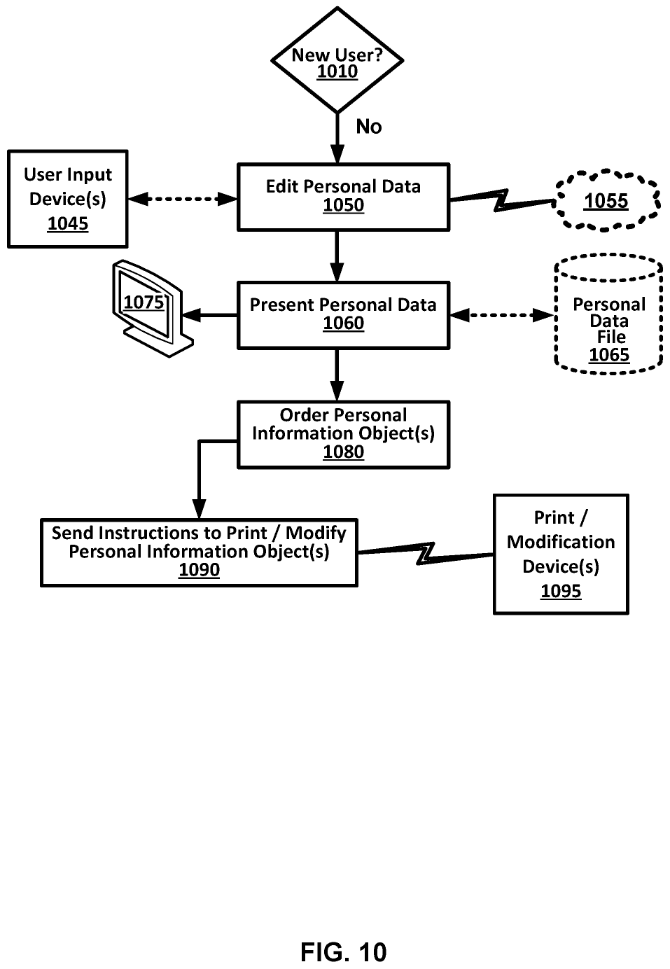

FIG. 10 is an example flow diagram of producing a personal information object as per an aspect of some of the various embodiments. Producing a personal information object may comprise determining if a user is a new user or an existing user at block 1010. Producing a personal information object may comprise enabling returning users to edit personal data at block 1050. At least one User Input Device 1045 may be employed to edit the personal data. At least a portion of the personal data may be downloaded from a remote device. The remote device may be associated with Cloud 1055. For example, a cloud may comprise an internet. The Internet, a network of devices, combinations thereof, and/or the like. Producing a personal information object may comprise presenting personal data at block 1060. At least a portion of the personal data may be presented on Display 1075. At least a portion of the personal data may be stored in a Personal Data File 1065. The personal data file may, for example, be stored on a user device, on a detachable storage drive, in a personal data store located on at least one remote device, combinations thereof, and/or the like. Producing a personal information object may comprise accepting at least one order for at least one personal information object at block 1080. Producing a personal information object may comprise sending instructions to print a personal information card and/or modify a personal information article at block 1090. The instructions may be sent to at least one Print/Modification Device 1095.

Persons skilled in the art(s) will recognize that producing a personal information object encoded with personal data for use as a patient in an emergency or medical situation is only one example of many uses for encoding personal data on a personal information object. Other examples of personal data may include but are not limited to college transcripts, a plurality of camp activities, a schedule associated with a plurality of camp activities, a plurality of classes, a schedule associated with a plurality of classes, a plurality of meetings, a schedule associated with a plurality of meetings, a plurality of conference presentations, a schedule associated with a plurality of conference presentations, personal financial information, mortgage information, pets owned by the user, combinations thereof, and/or the like. Other uses for a personal information object may include but are not limited to personal identification, credentials, access to a secure facility, combinations thereof, and/or the like.

According to some of the various embodiments, the computer readable instructions may be configured to cause at least one processor to receive personal data associated with an anticipated owner of a personal information article. By way of example and not limitation, the anticipated owner may be a user of the computer readable instructions attempting to order and/or modify a personal information article. Alternatively, the user may be attempting to order and/or modify a personal information article for at least one other person.

According to some of the various embodiments, the computer readable instructions may be configured to cause at least one processor to present at least a portion of the personal data on a display. The computer readable instructions may also be configured to cause at least one processor to obtain a signature from a user for authorization of at least one of the declaration(s). The declaration(s) may be associated with the anticipated owner. At least one of the declaration(s) may comprise a reference. The reference may be designated by the anticipated owner. The computer readable instructions may be further configured to present an option to cause the personal information article to be modified to comprise at least one barcode. The computer readable instructions may also be configured to cause at least one additional personal information article to be modified.

According to some of the various embodiments, the computer readable instructions may be configured to cause at least one processor to present at least one user interaction item. The user interaction item may be configured to present emergency contact information, health care provider information, insurance provider information, relative contact information, financial donation information, donor information, order information for at least one personal information article, combinations thereof, and/or the like.

According to some of the various embodiments, the personal information article may be modified via a printing device, a stamping device, an engraving device, an etching process, a lithographic device, a heat transfer device, combinations thereof, and/or the like. The printing device may comprise a 3-D printer. The computer readable instructions may be further configured to cause at least one processor to enable the user to decline the modification of the personal information article.

According to some of the various embodiments, the personal information article may comprise a wristband, a necklace, a dog tag, a choker, a key chain, a case, a heat transferable material, a wearable item, a piece of jewelry, combinations thereof, and/or the like. An example of a case is a mobile device case. An example of a heat transferable material is heat transfer paper. The personal information article may comprise paper, metal, wood, bamboo, rubber, at least one synthetic material, combinations thereof, and/or the like.

According to some of the various embodiments, the personal information article may be modified to comprise a declaration(s). The declaration(s) may comprise a waiver of privacy rights, an advanced directive declaration, a do not resuscitate declaration, an Allow a Natural Death declaration, a do not incubate declaration, combinations thereof, and/or the like. The personal information article may further be modified to comprise a signature of the anticipated owner. The personal information article may also be modified to comprise a biometric identifier of the anticipated owner. The personal information article may be modified to comprise a photo of the anticipated owner. The modification of the personal information article may be specific to a state or local jurisdiction. The personal information article may be modified to comprise article information. The article information may comprise a barcode date, a modification date, a symbol representing a portion of the personal data, an acronym representing a portion of the personal data, combinations thereof, and/or the like.

According to some of the various embodiments, the at least one barcode may be formatted as a Quick Response (QR) code, a Portable Data File (PDF) 417 barcode, a matrix barcode, and/or the like. The at least one barcode may comprise a three-dimensional relief. The at least one barcode may further comprise an encoded signature. The at least one barcode may further comprise at least one hyperlink and/or network address representing a network device configured to accept notifications each time at least one of the at least one barcode is scanned. The notification may be immediate or delayed. The notification may be routed through at least one network device associated with a provider of a personal information card or personal information article. The notification may require authentication.

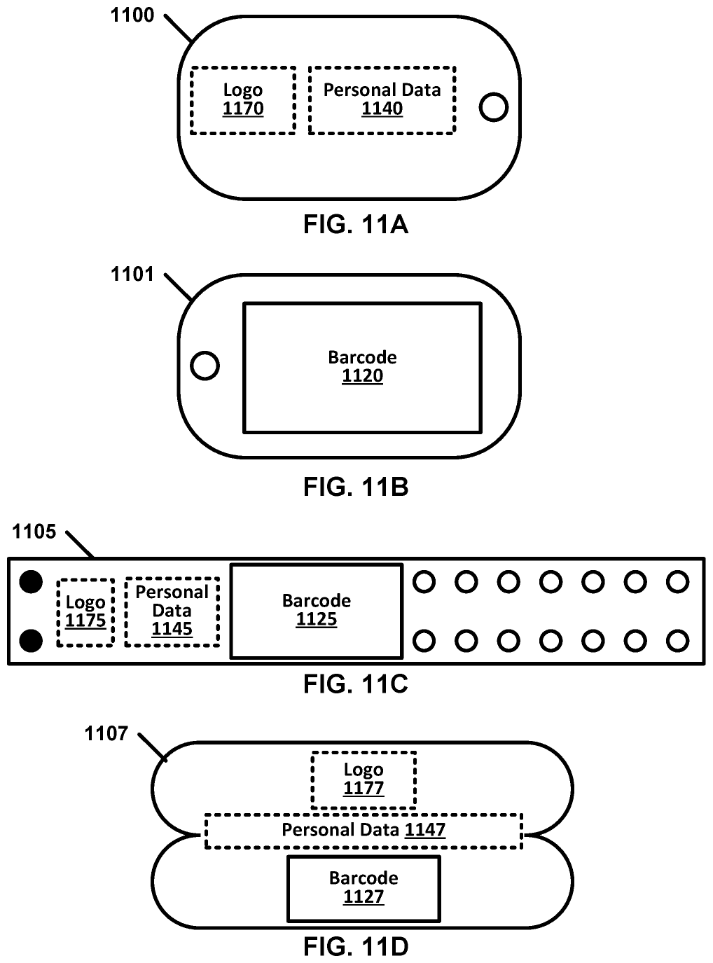

FIG. 11A and FIG. 11B illustrate two sides of an example personal information article as per aspects of some of the various embodiments. A first side 1100 of a personal information article is illustrated in FIG. 11A. The first side 1100 may comprise at least a portion of Personal Data 1140. The first side 1100 may comprise at least one Logo 1170. A second side 1101 of a personal information article is illustrated in FIG. 11B. The second side 1101 may comprise at least one Barcode 1120.

FIG. 11C illustrates an example personal information article as per aspects of some of the various embodiments. Personal information article 1105 may comprise a wristband. Personal information article 1105 may comprise at least a portion of Personal Data 1145. Personal information article 1105 may comprise at least one Logo 1175. Personal information article 1105 may comprise at least one Barcode 1125.

FIG. 11D illustrates an example personal information article as per aspects of some of the various embodiments. Personal information article 1107 may comprise a patch. Personal information article 1107 may comprise at least a portion of Personal Data 1147. Personal information article 1107 may comprise at least one Logo 1177. Personal information article 1107 may comprise at least one Barcode 1127.

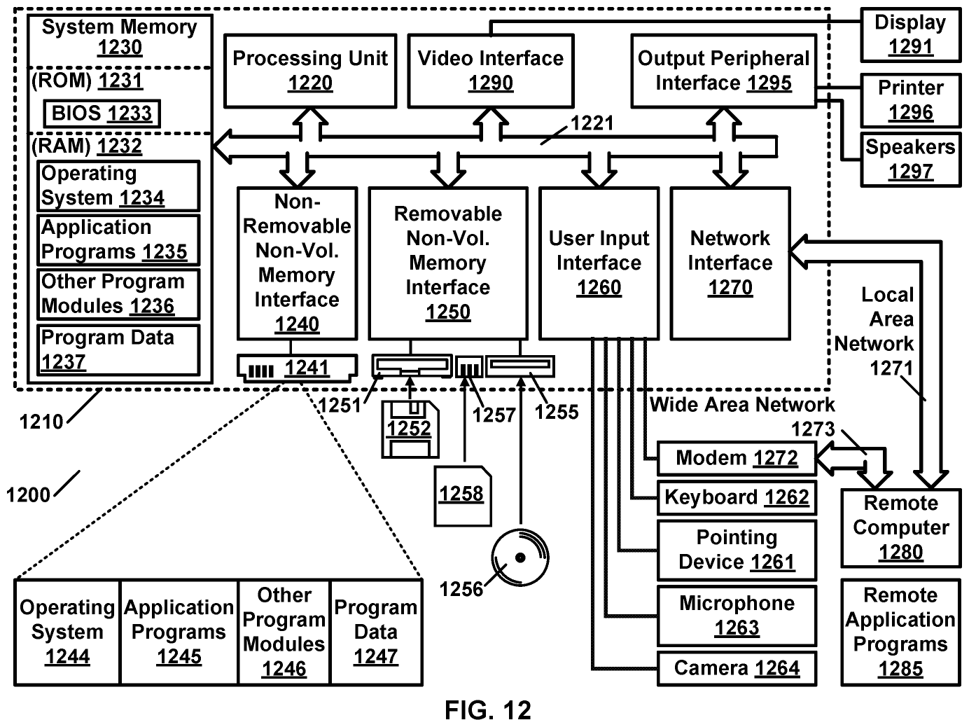

FIG. 12 illustrates an example of a suitable computing system environment 1200 on which aspects of some embodiments may be implemented. The computing system environment 1200 is only one example of a suitable computing environment and is not intended to suggest any limitation as to the scope of use or functionality of the claimed subject matter. Neither should the computing environment 1200 be interpreted as having any dependency or requirement relating to any one or combination of components illustrated in the exemplary operating environment 1200.

Embodiments are operational with numerous other general purpose or special purpose computing system environments or configurations. Examples of well-known computing systems, environments, and/or configurations that may be suitable for use with various embodiments include, but are not limited to, embedded computing systems, personal computers, server computers, hand-held or laptop devices, multiprocessor systems, microprocessor-based systems, set top boxes, programmable consumer electronics, network PCs, minicomputers, mainframe computers, telephony systems, distributed computing environments that include any of the above systems or devices, and the like.

Embodiments may be described in the general context of computer-executable instructions, such as program modules, being executed by a computer. Generally, program modules include routines, programs, objects, components, data structures, etc. that perform particular tasks or implement particular data types. Some embodiments are designed to be practiced in distributed computing environments where tasks are performed by remote processing devices that are linked through a communications network. In a distributed computing environment, program modules are located in both local and remote computer storage media including memory storage devices.

With reference to FIG. 12, an example system for implementing some embodiments includes a general-purpose computing device in the form of a computing device 1210. Components of computing device 1210 may include, but are not limited to, a processing unit 1220, a system memory 1230, and a system bus 1281 that couples various system components including the system memory to the processing unit 1220.

Computing device 1210 typically includes a variety of computer readable media. Computer readable media may comprise any available media that may be accessed by computing device 1210 and may include both volatile and nonvolatile media, removable and non-removable media. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, random access memory (RAM), read-only memory (ROM), electrically erasable programmable read-only memory (EEPROM), flash memory or other memory technology, compact disc read-only memory (CD-ROM), digital versatile disks (DVD) or other optical disk storage, magnetic cassettes, magnetic tape, magnetic disk storage or other magnetic storage devices, or any other medium which may be used to store the desired information and which may be accessed by computer 1210. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency (RF), infrared and other wireless media. Combinations of any of the above should also be included within the scope of computer readable media.

The system memory 1230 includes computer storage media in the form of volatile and/or nonvolatile memory such as ROM 1231 and RAM 1232. A basic input/output system 1233 (BIOS), containing the basic routines that help to transfer information between elements within computer 1210, such as during start-up, is typically stored in ROM 1231. RAM 1232 typically contains data and/or program modules that are immediately accessible to and/or presently being operated on by processing unit 1220. By way of example, and not limitation, FIG. 12 illustrates operating system 1234, application programs 1235, other program modules 1236, and program data 1237.

The computing device 1210 may also include other removable/non-removable volatile/nonvolatile computer storage media. By way of example only, FIG. 12 illustrates a hard disk drive 1241 that reads from or writes to non-removable, nonvolatile magnetic media, a magnetic disk drive 1251 that reads from or writes to a removable, nonvolatile magnetic disk 1252, a flash drive reader 1257 that reads flash drive 1258, and an optical disk drive 1255 that reads from or writes to a removable, nonvolatile optical disk 1256 such as a CD ROM or other optical media. Other removable/non-removable, volatile/nonvolatile computer storage media that may be used in the operating environment include, but are not limited to, magnetic tape cassettes, flash memory cards, digital versatile disks, digital video tape, solid state RAM, solid state ROM, and the like. The hard disk drive 1241 is typically connected to the system bus 1281 through a non-removable memory interface such as interface 1240, and magnetic disk drive 1251 and optical disk drive 1255 are typically connected to the system bus 1281 by a removable memory interface, such as interface 1250.

The drives and their associated computer storage media discussed above and illustrated in FIG. 12, provide storage of computer readable instructions, data structures, program modules and other data for the computer 1210. In FIG. 12, for example, hard disk drive 1241 is illustrated as storing operating system 1244, application programs 1245, program data 1247, and other program modules 1246. Additionally, for example, non-volatile memory may include instructions for presenting and updating personal data on a display 1291 of computing device 1200 and/or the like. Similarly, non-volatile memory may include instructions for causing the presentation of personal data on the display of a remote computing device 1280 and/or the like.

A user may enter commands and information into computing device 1210 through input devices such as a keyboard 1268, a microphone 1263, a camera 1264, and a pointing device 1261, such as a mouse, trackball or touch pad. These and other input devices are often connected to the processing unit 1220 through a user input interface 1260 that is coupled to the system bus, but may be connected by other interface and bus structures, such as a parallel port, game port or a universal serial bus (USB). A display 1291 or other type of display device may also be connected to the system bus 1281 via an interface, such as a video interface 1290. Other devices such as, for example, speakers 1297 and printer 1296, may be connected to the system via peripheral interface 1295.

The computing device 1210 may be operated in a networked environment using logical connections to one or more remote computers, such as a remote computer 1280. The remote computer 1280 may be a personal computer, a hand-held device, a server, a router, a network PC, a peer device or other common network node, and typically includes many or all of the elements described above relative to the computing device 1210. The logical connections depicted in FIG. 12 include a local area network (LAN) 1271 and a wide area network (WAN) 1273, but may also include other networks. Such networking environments are commonplace in offices, enterprise-wide computer networks, intranets and the Internet.

When used in a LAN networking environment, the computing device 1210 is connected to the LAN 1271 through a network interface or adapter 1270. When used in a WAN networking environment, the computing device 1210 typically includes a modem 1272 or other means for establishing communications over the WAN 1273, such as the Internet. The modem 1272, which may be internal or external, may be connected to the system bus 1281 via the user input interface 1260, or other appropriate mechanism. The modem 1272 may be wired or wireless. Examples of wireless devices may comprise, but are not limited to: Wi-Fi and Bluetooth. In a networked environment, program modules depicted relative to the computing device 1210, or portions thereof, may be stored in the remote memory storage device. By way of example and not limitation, FIG. 12 illustrates remote application programs 1285 as residing on remote computer 1280. It will be appreciated that the network connections shown are presented as examples only and other means of establishing a communications link between the computers may be used.

The subject matter includes personal data associated with a card holder. It is to be understood that the term “associated” is to be interpreted as related to. For example, a portion of the personal data may be related to an attribute of the card holder.

The subject matter also includes at least one barcode encoded to contain at least a portion of personal data. By way of example and not limitation, the personal data encoded in a barcode may be transmitted to a computing device managed by a first responder, an emergency medical technician, a medical staff member, and/or the like. The personal data may be transmitted, for example, by scanning the barcode with a barcode reader, a barcode scanner, a camera, combinations thereof, and/or the like. Barcodes may be scanned from a display, a personal information card, a personal information article, combinations thereof, and/or the like. Persons skilled in the art will recognize that other methods may be employed for transmitting personal data from a personal information card and/or personal information article to a computing device such as reading an embedded NFC element, a radio frequency identification (RFID) chip, a contactless smartcard, a card or chip that requires electrical contact, combinations thereof, and/or the like. The element, card, or chip may be passive and/or active. Alternatively, at least a portion of personal data may be transmitted directly from a first wireless computing device to a second wireless computing device. Communication between wireless computing devices may employ at least one wireless protocol such as Bluetooth, Wi-Fi, NFC, cellular communications, combinations thereof, and/or the like. Communication between wireless computing devices may employ at least one barcode and at least one scanner.

The subject matter also includes computer readable instructions configured to cause at least one processor to enable a user to order a personal information card and/or personal information article. By way of example and not limitation, the computer readable instructions may be configured to cause communication via communications hardware with a third party payment processor, a third party merchant exchange carrier, a bank, an electronic commerce service provider, combinations thereof, and/or the like.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

In this specification, “a” and “an” and similar phrases are to be interpreted as “at least one” and “one or more.” References to “an” embodiment in this disclosure are not necessarily to the same embodiment.

Many of the elements described in the disclosed embodiments may be implemented as modules. A module is defined here as an isolatable element that performs a defined task and has a defined interface to other elements. The modules described in this disclosure may be implemented in hardware, a combination of hardware and software, firmware, wetware (i.e. hardware with a biological element) or a combination thereof, all of which are behaviorally equivalent. For example, modules may be implemented using computer hardware in combination with software routine(s) written in a computer language (Java, HTML. XML. PHP, Python, ActionScript, JavaScript, Ruby, Prolog. SQL, VBScript, Visual Basic. Perl, C, C++. Objective-C or the like). Additionally, it may be possible to implement modules using physical hardware that incorporates discrete or programmable analog, digital and/or quantum hardware. Examples of programmable hardware include: computers, microcontrollers, microprocessors, application-specific integrated circuits (ASICs), field programmable gate arrays (FPGAs), and complex programmable logic devices (CPLDs). Computers, microcontrollers and microprocessors are programmed using languages such as assembly, C. C++, and/or the like. FPGAs. ASICs and CPLDs are often programmed using hardware description languages (HDL) such as VHSIC hardware description language (VHDL) or Verilog that configure connections between internal hardware modules with lesser functionality on a programmable device. Finally, it needs to be emphasized that the above mentioned technologies may be used in combination to achieve the result of an operational module.

Some embodiments may employ processing hardware. Processing hardware may include one or more processors, computer equipment, embedded systems, machines, and/or the like. The processing hardware may be configured to execute instructions. The instructions may be stored on a machine-readable medium. According to some embodiments, the machine-readable medium (e.g. automated data medium) may be a medium configured to store data in a machine-readable format that may be accessed by an automated sensing device. Examples of machine-readable media include: magnetic disks, cards, tapes, and drums, flash memory, memory cards, electrically erasable programmable read-only memory (EEPROM), solid state drives, optical disks, barcodes, magnetic ink characters, and/or the like.

While various embodiments have been described above, it should be understood that they have been presented by way of example, and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. Thus, the present embodiments should not be limited by any of the above described exemplary embodiments. In particular, it should be noted that, for example purposes, the computer readable instructions may reside on a mobile device and/or at least one server. Present embodiments may be presented, according to some of the various embodiments, through at least one webpage, on a mobile device, through an Application Programming Interface (API), combinations thereof, and/or the like. However, one skilled in the art will recognize that the server and mobile device may vary from a traditional server/device relationship over a network such as the internet. For example, a server may be collective based: portable equipment, broadcast equipment, virtual, application(s) distributed over a broad combination of computing sources, part of a cloud, and/or the like. Similarly, for example, a mobile device may be a user based client, portable equipment, broadcast equipment, virtual, application(s) distributed over a broad combination of computing sources, part of a cloud, and/or the like. A mobile device may comprise a small, lightweight, portable computing device, typically, but not always, having a display screen, an input device, and various sensors. Some mobile devices are configured to perform specific functions, such as, for example, emergency reporting/management tasks. Examples of mobile device(s) comprise, but are not limited to: cell phones, tablets, eReaders, wearable electronics, laptops, recording devices, scanning devices, prompting devices, combinations thereof, and/or the like. Additionally, it should be noted that, for example purposes, several of the various embodiments were described as modules. However, one skilled in the art will recognize that many various languages and frameworks may be employed to build and use embodiments of the present invention. For example, languages/frameworks may be based upon C. C++. Objective-C, Linux, Java, Metro, Modern, combinations thereof, and/or the like.

In addition, it should be understood that any figures that highlight any functionality and/or advantages, are presented for example purposes only. The disclosed architecture is sufficiently flexible and configurable, such that it may be utilized in ways other than that shown. For example, the steps listed in any flowchart may be re-ordered or only optionally used in some embodiments.

Further, the purpose of the Abstract of the Disclosure is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract of the Disclosure is not intended to be limiting as to the scope in any way.

Finally, it is the applicant's intent that only claims that include the express language “means for” or “step for” be interpreted under 35 U.S.C. 112. Claims that do not expressly include the phrase “means for” or “step for” are not to be interpreted under 35 U.S.C. 112.

1-20. (canceled)

21. A personal information card, comprising:
a two-dimensional barcode encoded to contain personal data associated with a card holder, the personal data including at least one of a waiver of privacy rights, an advanced directive declaration, an Allow a Natural Death (AND) declaration, a Do Not Resuscitate (DNR) declaration, and a Do Not Incubate (DNI) declaration; and
a signature from the card holder authorizing the use of the at least one declaration, the signature encoded in the two-dimensional barcode, and the signature being a written signature, an encoded signature or a reference to a signature.

22. The personal information card of claim 21 further comprising an RFID chip or near field communication device.
